(19) United States
(12) Patent Application Publication
(10) Pub. No.: US 2018/0345479 A1
(43) Pub. Date: Dec. 6, 2018

(54) ROBOTIC COMPANION DEVICE

(71) Applicants: Rocco MARTINO, Villanova, PA (US); Joe MARTINO, Villanova, PA (US)

(72) Inventors: Rocco MARTINO, Villanova, PA (US); Joe MARTINO, Villanova, PA (US)

(21) Appl. No.: 15/995,943

(22) Filed: Jun. 1, 2018

Related U.S. Application Data
(60) Provisional application No. 62/514,799, filed on Jun. 3, 2017.

(51) Int. Cl. B25J 9/00; B25J 9/16; B25J 5/00; B25J 19/02; G16H 50/50; G16H 50/80; G16H 50/20; G16H 80/00; G16H 15/00

(52) U.S. Cl. CPC B25J 9/0003; B25J 9/1697; B25J 5/007; B25J 19/023; G16H 15/00; G16H 50/80; G16H 50/20; G16H 80/00; G16H 50/50

(57) ABSTRACT

Embodiments described herein relate to a robotic companion device for assisting a patient with daily tasks and/or medical emergencies. A robotic companion device may have sensors, actuators, and a computer system. The robotic device maybe able to receive sensor data, such as environmental information and a patient location. As the robotic device receives more data, it can learn (i.e., update decision tables) from the environment or patient in order to guide future actions. Additionally, based on the received sensor data, the robotic device is able to determine whether an emergency situation has occurred related to one or more patients and move to an area adjacent to the patient. Once near the patient, the robotic device can transmit a communication to a third party alerting them of the emergency situation. To improve patient interactions, the robotic device may mimic the gender, voice, lip movement, or facial appearance of a familiar individual.

### CROSS-REFERENCE TO RELATED APPLICATIONS

The present application claims benefit of priority under 35 U.S.C. 119(e) to the filing date of U.S. Provisional Patent Application 62/514,799 filed Jun. 3, 2017, entitled, “ROBOTIC COMPANION DEVICE,” the content of which is incorporated herein by reference in its entirety.

### BACKGROUND

Personal robots are pre-programmed for use in personal and/or household applications. The robot-to-human interface in personal robots is designed so any human being, even with little or no robotic knowledge, can operate these robots easily and usefully. The aging population globally is driving the growing demand in the personal robot industry for companion robots.

Generally, robotic research platforms have not been designed to consider a home environment or personal preferences and concerns (e.g., making the robot non-intrusive, familiar, and welcoming). Although robotic systems have been developed to assist caregivers in various actions (e.g., carrying medications, providing amusing interaction, providing communication means, etc.), there is an unmet need for smaller more intuitive robotic assistance for the patients themselves in a home-like environment.

The lack of appropriately skilled companion robot designs, and the technical complexity of robot-to-human interfaces have hindered the growth of critical companion robot applications, such as assisting the elderly monitor their health daily. There are many other activities that a companion robot may be able to perform with human beings, as discussed further herein.

### SUMMARY

The present disclosure relates to a robotic companion device having: one or more sensors; one or more actuators; a network connection device; and a non-transitory, processor-readable storage medium that stores instructions executable by a processor to: receive, from the one or more sensors, sensor data, the sensor data comprising environmental information and patient location information, determine, based on the sensor data, that an emergency situation has occurred related to one or more patients, move, using the one or more actuators, the robotic companion device to an area adjacent to the one or more patients, wherein a path to the area is based on the patient location information and the environmental information, and transmit, using the network connection device, a communication to a third party.

Another embodiment relates to a robotic companion device having: one or more sensors; one or more actuators; and a non-transitory, processor-readable storage medium that stores instructions executable by a processor to: receive, from the one or more sensors, sensor data, the sensor data comprising environmental information and patient command information, the patient command information comprising a user input, wherein the user input is analyzed to determine that the user is a patient of one or more patients, determine, based on the patient command information, that an order has been given, move, using the one or more actuators, the robotic companion device to an area, wherein a path to the area is based on the environmental information, and perform the order given; wherein the robotic companion device is configured to visually imitate an individual known by the one or more patients.

A further embodiment relates to a system including: a robotic companion device having: one or more sensors, one or more actuators, a network connection device, a first power connector, and a non-transitory, processor-readable storage medium that stores instructions executable by a processor to: receive, from the one or more sensors, sensor data, the sensor data comprising environmental information and patient location information, determine, based on the sensor data, that an emergency situation has occurred related to one or more patients, move, using the one or more actuators, the robotic companion device to an area adjacent to the one or more patients, wherein a path to the area is based on the patient location information and the environmental information, and transmit, using the network connection device, a communication to a third party; and a base station having: a second power connector, and a power source, wherein the instructions are further executable by the processor to establish a connection between the first power connector and the second power connector, and wherein power is transferred from the power source to the robotic companion.

### BRIEF DESCRIPTION OF THE DRAWINGS

FIG. 1 depicts an illustrative embodiment of a robotic device.

FIG. 2 depicts an illustrative embodiment of a motorized seat or wheelchair.

FIG. 3 depicts an illustrative embodiment of a system comprising the robotic device and the motorized seat or wheelchair.

FIG. 4 depicts an illustrative embodiment of a telescoping motorized pedestal base.

FIG. 5 depicts an illustrative embodiment of a system comprising the robotic device and the motorized pedestal.

FIG. 6 depicts an illustrative embodiment of a motorized bipedal base.

FIG. 7 depicts an illustrative embodiment of a system comprising the robotic device and the motorized bipedal base.

FIG. 8 depicts a flow diagram illustrating an embodiment of operation of the robotic device.

FIG. 9 depicts various embodiments of a computing device for implementing the various methods and processes described herein.

### DETAILED DESCRIPTION

This disclosure is not limited to the particular systems, devices and methods described, as these may vary. The terminology used in the description is for the purpose of describing the particular versions or embodiments only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The following terms shall have, for the purposes of this application, the respective meanings set forth below.

As used herein, an electronic device or computing device refers to a device capable of receiving and processing one or more computer instructions to produce an output or other result. An electronic device includes a processing device and a tangible, computer-readable memory or storage device. The memory may contain programming instructions that, when executed by the processing device, cause the device to perform one or more operations according to the programming instructions. Illustrative examples of electronic devices or computing devices include personal computers, mobile devices, integrated circuits, and other similar devices designed and configured to perform one or more operations.

A robot or robotic device refers to a stand-alone system, for example, that is mobile and performs both physical and computational activities. The physical activities may be performed using a wide variety of movable parts including various tools or other similar end effectors, for example. The computational activities may be performed using a suitable processor and one or more computer readable memory devices, e.g., a data memory storage device. The computational activities may include processing information input from various sensors or other inputs of the robotic device to perform commanded functions; processing the input information, as well as other data in the memory stores of the robotic device, to generate a variety of desired information; or outputting information that has been acquired or produced by the robotic device to a desired destination, for example.

Disclosed herein are embodiments related to a system for interfacing with/helping/teaching human beings which includes a robotic device. It should further be understood that the terms: human, human being(s), person, patient, and/or individual may be used interchangeably throughout the specification and are in reference to an individual interacting with a robotic device. The system is designed to communicate with human beings via a built-in or held viewing screen, project images on a suitable surface via an integrated laser projector, or speak via the integrated speaker. These interactions (i.e., interfaces) can include things such as medical concerns and/or emergencies or simply everyday tasks. Embodiments may allow for the robot to access and interface with various devices possessed by or associated with a human. For example, the robot may be able to retrieve medical measurements (e.g., vital signs, blood pressure, temperature, oxygen saturation, etc.); access and use information in order to act as a companion (e.g., read digital books, articles, information or news, display digital videos/images, play music, etc.); facilitate communication to others (e.g., write letters, text messages, or emails, place telephone calls, etc.); assist with everyday tasks or errands (e.g., access the Internet for information or to purchase items, manage financial accounts, etc.); and educate (e.g., provide learning materials or audio/visual aid for disabled individuals).

The robotic device is intended to be a multi-functional familiar companion and friend to human beings. The device may have control interfaces for measurements from a group of sensors (e.g., proximity sensors, touch sensors, audio sensors, olfactory sensors, light sensors, visual sensors, etc.), which may respond to sensor inputs or previous learned inputs for moving the robotic device around or carrying out an action. Additionally, the robotic device may be able to do many other tasks beyond those listed herein, such as render assistance, call for assistance, put out fires, make coffee, tea or other refreshments, hold an individual's hand, sing to an individual, or perform any other task of comfort for an individual.

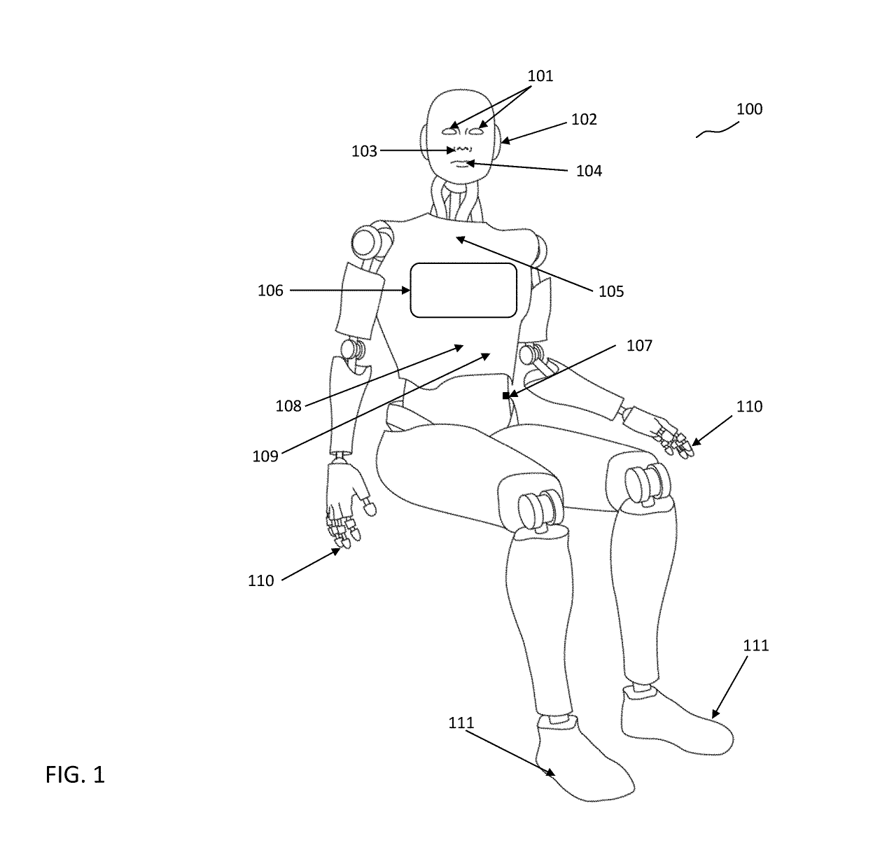

In order to carry out the various tasks discussed herein, various embodiments of the robotic device may comprise a plurality of sensors and/or interface options. For example, referring to FIG. 1, an embodiment of a robotic device 100 may have visual and/or optical sensors 101, which may, as shown, be located where a human's eyes would be. In some embodiments, laser and/or light projectors may also be included. As discussed herein, these allow the robotic device to project images as well as measure distances. In additional embodiments, the robotic device may have audio sensors 102, olfactory sensors 103, touch sensors 110, and proximity sensors 111. It should be noted that the locations identified in FIG. 1 are for exemplary purposes only, and that a sensor's location may vary based on the robot's size and shape, as well as its primary function.

In further embodiments, the robotic device may also have one or more integrated speakers 104 to play audio sounds and/or to communicate with a patient. The robotic device may also have a network communication device 105 to enable it to communicate with other devices and subsystems (e.g., local devices, remote devices, etc.). The network communication device may be a wired connection (e.g., a connection that takes place during charging or docking) or a wireless connection. It should be understood, that the wireless connection may use any form of wireless communication, such as for example, satellite communication, infrared communication, radio frequency communication, microwave communication, Wi-Fi communication, cellular communication, Bluetooth communication, etc.

As further discussed herein, the robotic device may also comprise a display or touch display 106. The display may be use used to display various types of media or information to a patient, as further discussed herein. In addition, the robotic device may have various other technical features, such as, for example, a computer system (e.g., processor, memory, storage medium, etc.) 108, connection ports (e.g., serial, USB, SATA, eSATA, SCSI, or any known means of connection) 107, and one or more microcontrollers and/or servo boards 109.

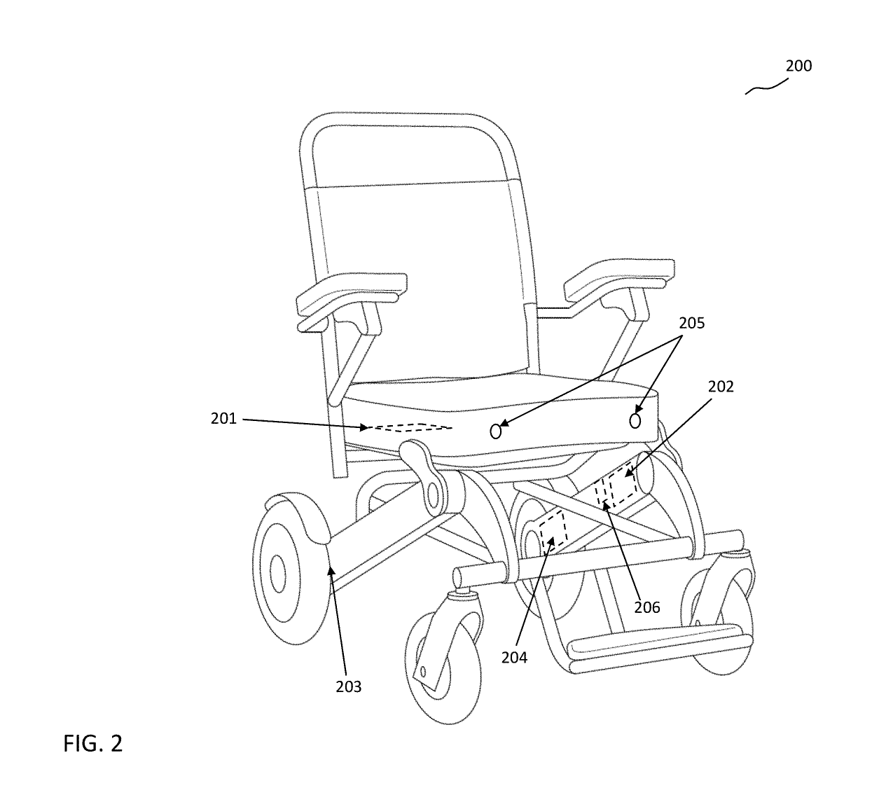

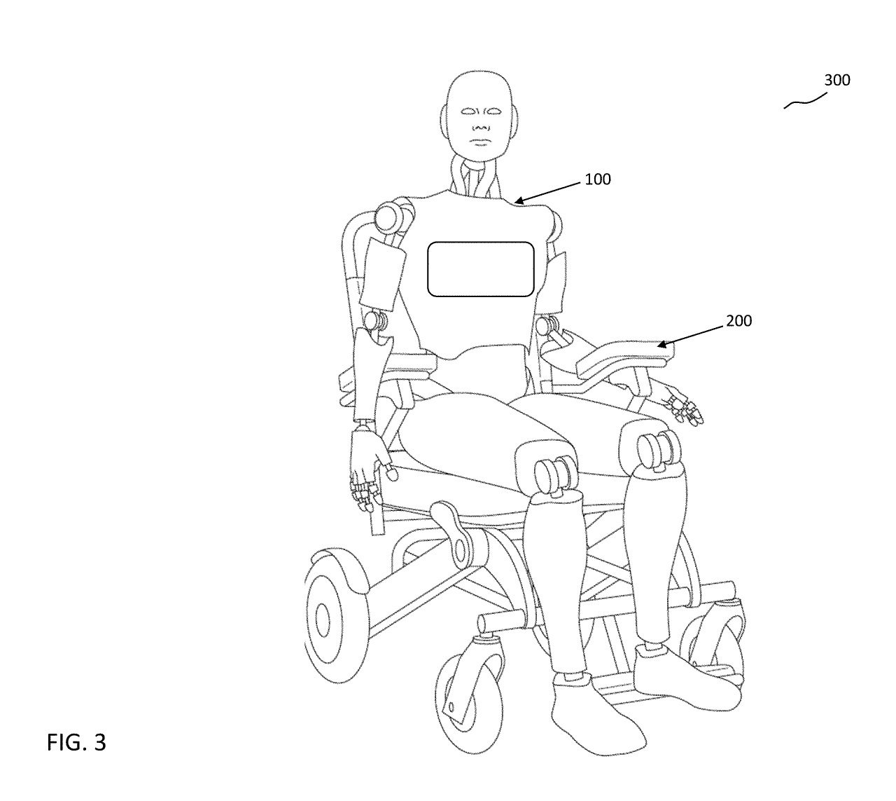

In some embodiments, the robotic device may be in a seated position. This may make the robotic device seem more natural. As shown in FIG. 2, a mobile chair or wheelchair device 200 may be utilized to enable the movement of the robotic device. As shown, and further discussed herein, the chair 200, may comprise a motor controller and relay boards 201, a battery charging port and/or AC/DC converter 202, a wheel motor mount 203, one or more rechargeable battery packs 204, proximity sensors (e.g., in the front and back) 205, and a voltage regulator 206. Accordingly, as shown in FIG. 3, in some embodiments, the system 300 may comprise the robotic device 100 in combination with the wheelchair device 200.

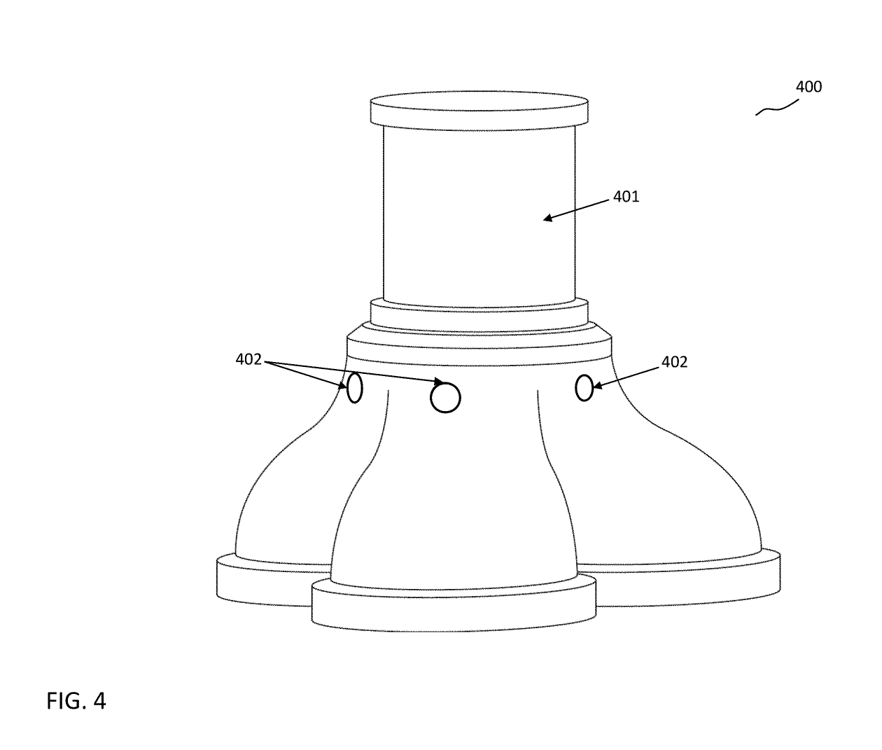

In additional embodiments, the robot may stand upright. As will be discussed with reference to FIGS. 4-7, various implementations may be utilized with regard to the vertically oriented robotic device. For example, as shown in FIG. 4, in one embodiment, the robotic device may utilize a pedestal base 400. The pedestal base may connect to the robotic device via a vertical column 401. Similar to the robotic device itself, the vertical column 401 may be made out of any material of suitable strength and flexibility to adequately support the robotic device.

The pedestal base may comprise any form of motorized movement currently known or identified in the future that allows for rolling mobility. For example, the pedestal base may comprise one or more wheels (not shown) or one or more motorized balls (not shown), which may rotate in various directions, thus moving the pedestal. In a further embodiment, the vertical column 401 of pedestal base 400 may have the ability to raise and lower (i.e., be telescoping). This allows the robotic device 100 to raise and lower in order to perform the various tasks disclosed herein. In some embodiments the vertical column 401 may include one or more actuators (e.g., hydraulic actuators, pneumatic actuators, electric actuators, thermal actuators, magnetic actuators, or mechanical actuators).

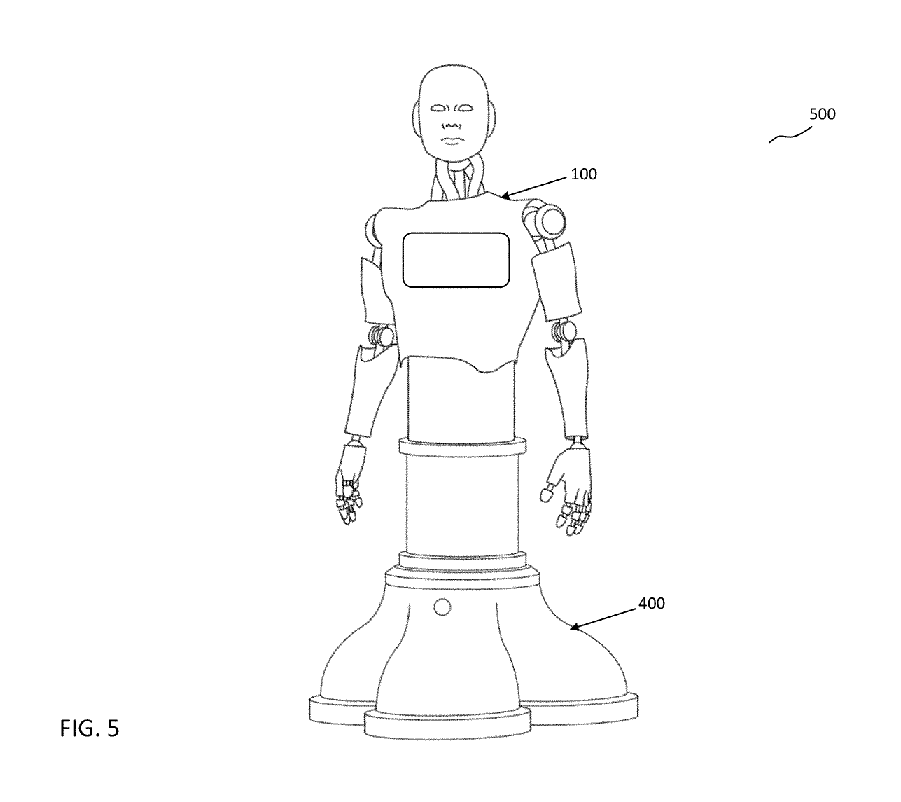

As shown in FIG. 4, the pedestal base may comprise one or more sensors 402. The sensors 402, may be any sensor as discussed herein with reference to the robotic device. In one or more embodiments, the sensors 402 may help the robotic device with movement and detection of objects in the environment. Accordingly, the sensors 402 may be located at various heights and positions on the robotic device. For example, one or more sensors 402 may be located at near floor level to detect potential collision objects. In further embodiments, the pedestal base may include sensors on the front, back, and sides in order to ensure it does not collide with or damage other objects. As shown in FIG. 5, the pedestal 400 may be attached to the robotic device 100 such that the proper center of gravity is achieved to allow the robotic device to easily move around a space.

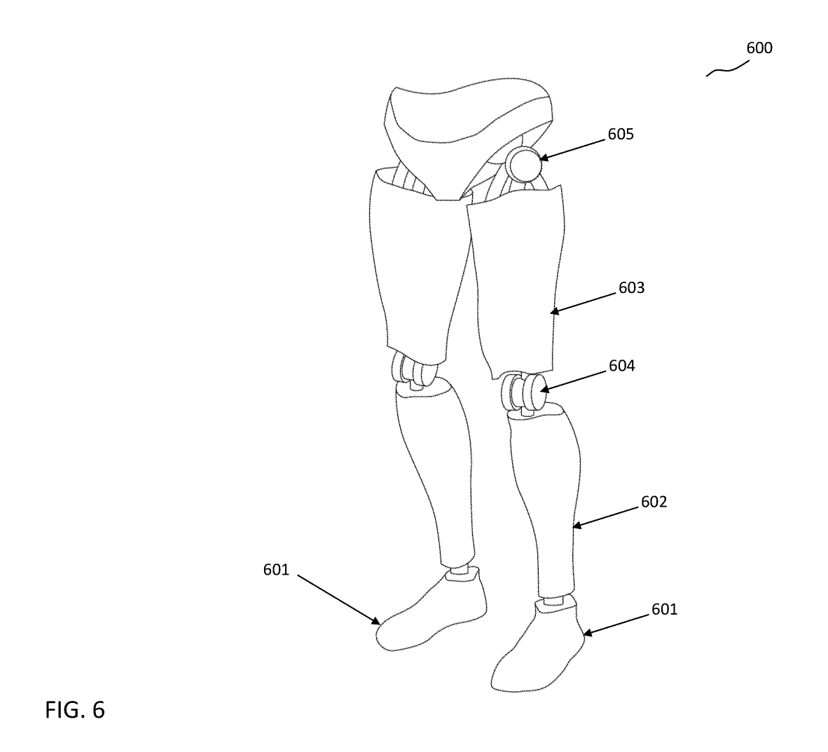

Referring now to FIG. 6, in some embodiments, the robotic device may have bipedal movement 600. As shown, the bipedal portion of the robotic device may comprise one or more base portions 601 (e.g., foot pads) which contact a surface (e.g., floor, stair, etc.) and allow the robotic device 100 to move around an environment. The base portions 601 may be rotatably attached to a lower leg portion 602, which may then be rotatably attached to an upper leg portion 603. As shown, the hinged connection 604 of the upper leg portion 603 and the lower leg portion 602, may allow for the robotic device to take one or more steps (i.e., walk). It should be noted that FIG. 6 is for exemplary purposes only, and that the bipedal nature of the robot may have multiple additional sections and additional hinged parts. Moreover, the bipedal base may have a plurality of limbs (i.e., two or more), and should not be limited to two limbs as shown.

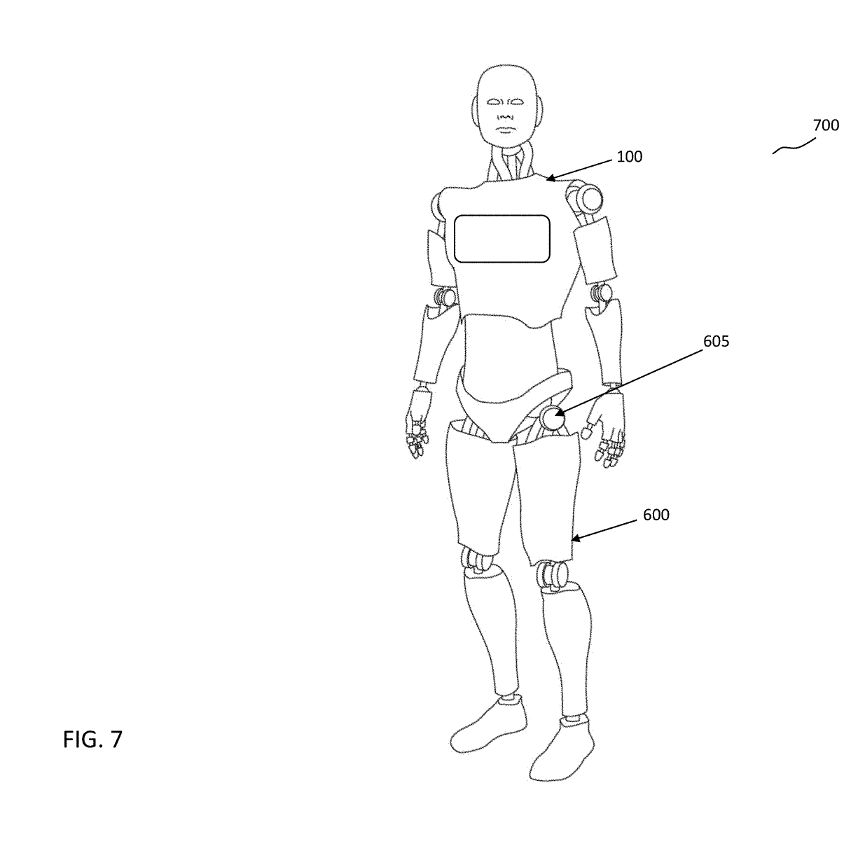

Referring now to FIGS. 6 and 7, a further embodiment is shown where the bipedal portion 600 of the robotic device 100 may have a connection point 605. The connection point 605 may be hinged such as to allow the robotic device to “bend,” thus mimicking a waist or hip of a human. Accordingly, in some embodiments, the robotic device 100 may pivot forward or backward with respect to the bipedal base 600. This may allow the robotic device to pick up objects on lower surfaces (e.g., the floor) or to gain access to the patient when they have fallen.

As discussed herein, some embodiments may comprise a robotic device which is intended to be a multi-functional familiar companion and friend to human beings and an interface for accepting commands from human beings. In addition, the robotic device may physically move within a defined area in response to voice or gestured commands issued by one or more individuals (e.g., medical patients, elderly patients, disabled patients, etc.). In one or more further embodiments, the robotic device may move and/or interact with people and objects based on information learned from human beings during previous interactions (e.g., historical knowledge) as well as the environment (e.g., the house, apartment, hospital room, living space, etc.).

In some embodiments, the robotic device may have a human-like appearance utilizing a synthetic silicon skin or any other material that may be durable and malleable to shape into an appearance associated with a person familiar to the patient using the robotic device. In some embodiments, the robotic device may be provided with an appearance of (i.e., visually imitate) a person familiar to the individual or patient using the robotic device. The robotic device may also be dressed or have the mannerisms of the familiar individual. If the robotic device is not configured to imitate someone familiar, it may still be customized to the patient's desires or the specific situation. For example, a robotic device that works only with elderly women may be configured to have a female appearance. Accordingly, there may be embodiments where the robotic device has the facial characteristics of either gender and may be dressed appropriately.

In further embodiments, the robotic device may sound like a specific person that is familiar to the patient. Moreover, the robotic device's lips may move in sync to a voice being produced from one or more speakers. Generally, the robotic device may have mobile capability, but it should be understood that, in some embodiments, a stationary version of a robotic device may be used. As discussed herein, the robotic device may appear as a human or mannequin sitting in a chair. This customizable feature allows the robotic device to be tailored to its intended purpose. For example, the robotic device may be fashioned as a non-threatening and reassuring middle-aged female with a kind, soft voice in order to be appealing to most elderly patients. The physical attributes of the gender, face, hair and outfit will be determined by the patient and/or environment.

As discussed, the physical attributes of the robotic device may be male or female in appearance, and the voice may be adjusted accordingly either during creation or after becoming operational. The hair style and hair color of the robotic device may be selected by the patient engaging with the unit. In some embodiments, the arms, hands, legs and torso of the robotic device may be detachable and/or simply for aesthetic purpose. In other embodiments, the hand of the robotic device may be highly sensitive to touch and/or heat, in the event that the patient would like to hold hands. In one non-limiting example, the body of the robotic device may be in a sitting position with the arms/hands resting in the lap of the robotic device. Alternatively, as discussed herein, the robotic device may be vertically oriented, having a pedestal or bipedal base. The robotic device may be made of fiberglass, synthetic silicon, or any other material that has sufficient durability for its intended use.

In some embodiments, the mobility of the robotic device may be incorporated into a chair or wheelchair. It should be understood, that the term chair and/or wheelchair are used herein for simplicity, but that any form of mobile transport may be used for the robotic device. As discussed and shown in FIG. 1, the robotic device may include an array of sensors, such as, proximity sensors, touch sensors, audio sensors, olfactory sensors, light sensors, and visual sensors for taking selected measurements. The eyes of the robotic device may include cameras utilized for mobility and communication. The robotic device may use instruments for taking measurements, such as, for example, a laser/conventional thermometer, blood pressure monitor, stethoscope, audio scope, laser range finder/ruler for distance measurements, an internal global positioning system (GPS) that may be beneficial in emergency situations, and other devices that the patient may have requested for daily engagements.

In some embodiments, the robotic device may act semi-autonomously (e.g., it may greet people) when entering a room, being in the home, or even at a store. In a further embodiment, the robotic device may offer various objects to a patient or guest (e.g., a cup of tea or coffee, refreshments, flowers, a gift, or some other inducement or greeting). In some embodiments, the semi-autonomous robotic device may respond to various prompts received from an array of sensors that take selected measurements by engaging in actions selected from a group of programmed or learned actions. For example, the robotic device may direct its eyes toward the source of a prompt, which may be the face of a patient.

It should be understood that the robotic device also includes one or more processors programmed to cause the robotic device to analyze sensor measurements to distinguish among types of physical contact with the specified patient. The robotic device may categorize the physical contact with another person or an object in the surrounding area as a different contact than with the specific patient. In an embodiment, the robotic device may carry on interactive conversations with the patient or one or more additional users (e.g., medical professionals, a guest, etc.).

In further embodiments, the system may include processors programmed to cause the robotic device to perform behaviors/tasks in response to physical contact with a specified human being, an unknown human being, or various objects in the surrounding area. This may include, for example, altering the direction that the robotic device is moving to avoid colliding with an object or person in the surrounding area. Additionally, it may simply halt the motion of the robotic device temporarily so that an obstacle may have time to alter its direction to avoid a possible collision. The robotic device may use data received via its array of sensors to calculate one or more alternate routes that it may use to reach the desired location in a surrounding area.

In some embodiments, the robotic device is programmed to function in several modes, including an autonomous robotic behavior mode in which actions of the robotic device are controlled by a specific decision table, which contains programmed actions in conjunction with gathered sensor data. The robotic device may also have a semi-autonomous robotic behavior mode in which a sequence of actions by the robotic device are initiated by spoken commands or gestures related to a specific place or object in the surrounding area.

In other embodiments, the robotic device may convey results or information by displaying them on a screen or touch screen panel (e.g., as shown in FIG. 1 and FIG. 3, the torso display). The screen may be integral to the robotic device or held by the robotic device. In further embodiments, the information may be presented via a projection onto a suitable surface via an incorporated laser projector, via an integrated speaker reading the information aloud, and/or visually using the robot itself (e.g., holding up a number of fingers to represent a numerical value).

Additionally, the system includes processors programmed to cause the robotic device to socialize/converse with the patient or other nearby individuals by accessing previous conversations or by utilizing information available on the Internet. Thus, in some embodiments, the robotic device may record and remember certain things said by a specific individual or the patient (e.g., family members' names, birth date(s), education history, anniversary's, etc.). Additionally, the robotic device may access information sources on the internet (e.g., recent news or weather information) in order to discuss relevant and topical subjects.

During regular use, the robotic device may need to recharge one or more battery packs such as 204. In some embodiments, the robotic device may self-charge, via a wired or non-wired (wireless) connection, or via a periodic attachment to a charging station that may house a quick charger for charging the onboard rechargeable batteries. The self-charging mechanism may be incorporated into various parts of the robotic device (e.g., the finger on the hand of the robotic device or the base of the robotic device). The robotic device may utilize an AC to DC convertor during the charging process.

In some embodiments, the system may include processors programmed to cause the robotic device to access the Internet for browsing, research, and/or e-commerce transactions based on input (e.g., spoken commands) by the patient. It should be understood that the selection of information may be by topic, article heading, news broadcast, current events, or any other type of information. The information may be displayed on the display, as discussed herein, projected onto a suitable surface via the integrated laser projector, and/or audibly transmitted via the integrated speaker.

The robotic device may also include processors programmed to cause the robotic device to audibly transmit any digital literature utilizing the integrated speaker to the specified human beings by accessing information available on the Internet or information used previously. The human beings' information may be saved or categorized by genre, title, author, and/or topic. In further embodiments, the robotic device may capture text (e.g. from the pages of a book, magazine, etc.) via the one or more image capture devices, use optical character recognition (OCR) or the like, and then the text emits via the integrated speaker. Thus, in some embodiments, the robotic device may audibly transmit existing books or magazines owned by the patient.

The robotic device may also include processors programmed to make use of big data (e.g., previously stored, downloaded via telecommunications access, available via the Internet, etc.). The stored information may be saved or categorized by recorded time, topic, genre, title, author, patient, etc.

In some embodiments, the robotic device may assist the patient in writing letters, emails, or text messages to send to others (e.g., family members, friends, etc.). For example, a contact list stored on the robotic device may be accessed. Alternatively, direct commands may be received from the patient. For example, if the contact does not exist, the patient may add contact information by speaking to the robotic device and commanding it to save the contact information. The information may be displayed on the screen via the display device, projected onto a suitable surface via the integrated laser projector, and/or audibly transmitted via the integrated speaker.

In other embodiments, the robotic device may assist a patient in accessing and displaying digital photographs, albums, and/or images for viewing purposes. For example, an image may be selected by receiving an auditory input form the patient identifying the name of a person, group and/or entity. It may also be possible in some embodiments to reference specific photographers, albums, and/or images.

Some embodiments may also assist the patient in accessing and displaying digital videos such as movies, television shows, and/or Flash files. A digital video may be selected by receiving an auditory input from the patient including a title/name of the movie, television show, and/or Flash file. The digital videos may also be selected from a list displayed by the robotic device. The movie, television show, and/or Flash file may be viewed on the in-torso or held touch screen panel, projected onto a suitable surface via the integrated laser projector, and/or audibly transmitted using the integrated speaker.

In other embodiments, the robotic device may audibly transmit digital music and/or audio files that. These digital music and/or audio files may be selected by receiving auditory input from the patient including the title/name of the digital music and/or audio file or may be selected from a list displayed by the robotic device. As discussed herein, the digital music and/or audio file may be audibly transmitted via the integrated speaker. In some embodiments, the robotic device may engage in sing-a-longs with the patient and/or accompany a patient on selected musical instruments (i.e., sing a duet).

It may also be possible for the robotic device to provide instruction to a patient. Information may be selected by the patient by speaking the topic, genre, title, and/or author of the information. Additionally, the information may be selected from a list displayed by the robotic device. The robotic device can play video, stored or recorded on any media; or access stored or recorded educational programs of any kind at any level. The information may be locally stored or retrieved from a remote location. Additionally, the robotic device may learn more about the patient with each interaction. As discussed herein, the learned behavior may be stored and utilized for future interactions.

In some embodiments, the robotic device may also medically assist the patient by recording vital signs and carrying out rudimentary physical examinations. The vital signs and data may be transmitted to a remote monitoring station and/or medical facility. Medical information may be gathered by the robotic device using various medical devices. For example, such information may be gathered by holding a stethoscope against different parts of the patient, shining a light which may be housed in the finger or another body part of the robotic device into the throat of the patient, holding an audioscope against the ear of the patient, and/or performing other medical tasks for the patient, such as monitoring blood pressure, taking a temperature, photographing an image of a skin irritation and emailing it to a doctor's office or remote medical facility for examination, etc. In some embodiments, the medical instrument may further comprise an electrocardiogram machine, ophthalmoscope, thermometer, stopwatch, or scale. In addition to medical devices operable by the robotic device, a patient may wear a medical tracking device which communicates with the robotic device via a wireless communication protocol, such as those discussed herein. In some embodiments, the wearable medical tracking device may measure a patient's temperature, blood pressure, pulse, oxygen saturation, etc.

The robotic device may perform additional tasks, such as rendering assistance, calling for assistance, putting out fires, making coffee, tea, or other refreshments, holding an individual's hand, singing to an individual, or performing any other task of comfort for an individual. These tasks may be performed using files that have been downloaded previously or by downloading new data (e.g., receiving a software update) from remote servers or via the Internet. Information received from these tasks may be viewed on the in-torso or held touch screen panel, projected onto a suitable surface via the integrated laser projector, and/or audibly transmitted via the integrated speaker.

As discussed herein, in some embodiments, a robotic device may include a computer system which controls all aspects of the robotic activity. For example, the computer system may control movement, speech, and how the robotic device understands the commands of a patient. In some embodiments, the commands may be parsed from speech, received via electronic transmission, or selected from a set of commands contained in the robot's internal programming. It may also be possible for commands to be stored in the robot's non-active database, or stored remotely (i.e., in the cloud).

Accordingly, as discussed herein, a robotic device may include a mannequin-like figure. In various embodiments, the robotic device may be humanoid in appearance or not, it may resemble a male or female, and it may appear to be of a certain ages and appearances (e.g., formal dress, casual dress, relaxed dress, etc.). In a further embodiment, the lips may or may not quiver/move in synchronization with speech (e.g., audio emitted from the internal speaker), and the eye sockets may be used for a camera and/or projector device. In one embodiment, one eye location may be used for a camera, and the other may be used for a projector lens.

With regard to the movement of the robotic device, in some embodiments, the system may incorporate a Global Position System (GPS) or similar capability. In a further embodiment, the patient may also have a wearable or micro device on their body that is GPS enabled (i.e., reception and broadcast ability). Thus, in some embodiments, the robotic device may communicate with a patient's wearable device to determine the location and distance from its primary target (e.g., the patient). For example, a patient equipped with a micro device, which is in electronic communication with the robot, may automatically and continuously broadcast its position and vital signs on demand or as scheduled. In a further embodiment, the robotic device may be used as a communication bridge that transmits the data from the wearable device to a third party (i.e., a medical professional, one or more persons, and/or one or more control desks manned by persons or robots).

In a further embodiment, the robotic device can interpret commands received from its auditory sensors. For example, the robotic device may comprise a control program which can parse sentences spoken (e.g., by the patient or other individual) to the robot and determine any and all keywords indicating a command. In some embodiments, each keyword may be linked to a database via the keyword acting as identification for a record in the database that contains customized or specific actions or commands. The database identifies the record which contains a sound recording or keyword that will be executed by the robotic device together with additional directions and/or context provided by the patient.

If two or more commands are identified, the robotic device may audibly transmit (i.e., play audio through the integral speaker) to ask for the patient to select from a short list of keyword commands parsed from the original statement. Once the proper command has been identified via patient input, the robotic device may execute the order. In some embodiments, this is carried out by the correct record being sent to the processor in the robotic device for execution. As discussed herein, when the robot speaks (i.e., plays audio over the speaker) the lips may or may not quiver/move in synchronization with the words spoken. Once verbally stated, the robotic device may then execute the command.

In additional embodiments, the robotic device may have a control program that controls the movement of the robotic device from a first original location to where it is required to move to execute the command. For example, if the patient says, “come here,” then the robotic device may move to the patient location. The pathing and relative distance to the patient may be determined based on the environment and/or order given. For example, the patient may have different needs depending on whether are in bed, in a chair, in the bathroom, or wherever.

In some embodiments in order to expedite the movement of the robotic device, a map of the room and any anterior sites may be created and stored in a database for each patient. Thus, if a robotic device serves multiple individuals, it may have mapping and environmental data associated with each of the individuals. Thus, in some embodiments, the robotic device may authenticate one or more patients based on an input (e.g., a pin code, password, voice recognition, iris scan, etc.). Once a specific user or patient has been identified, the map and environmental data for that user or patient is loaded.

In addition to companionship, the robotic device may offer emergency care or alerts. For example, if the robotic device detects a “thud” or loud noise not normally heard, it may indicate that a patient has fallen. Thus, in some embodiments, once an external sound or trigger is detected, the robotic device may go into an emergency mode. For example, the robotic device may attempt to locate the patient and immediately go to their location. Once at the location of the patient and/or the sound of impact, the robotic device may determine whether the patient needs help and if so it may broadcast that requirement together with the location of the patient to a third party (e.g., a nurse or aid at a control desk, an emergency dispatcher such as 911, etc.). In a further non-limiting example, the robotic device may detect that the patient is attempting to get up. Based on this determination, the robot may tell the patient to stay where they are until help arrives. The robotic device may provide updates on when help is expected to arrive, or it may act as a communication bridge between the patient and the third party. In some embodiments, the robotic device will continue to issue reassuring statements and request the patient to stay put.

In some embodiments, the robotic device may be controlled by a computer regulated by a multifaceted program the parts of which are: (a) a command control module, which regulates the robotic device's response to commands it receives verbally or that are parsed in order to locate the identification which it will use to find the associated command and retrieve the database record; (b) speech interpretation and parsing of keywords; (c) speech synthetization; and (d) vocabulary control (i.e., enlargement of the command structure by adding new commands; in the event that the parsed command cannot be found in the database, a query will be issued to the patient or a third party for clarification; if the parsed command sentence leads to two or more commands, a query will be issued to the patient or a third party for clarification; in the database record for each command, a statement is included). In a further embodiment, the program has a section dedicated to execution of each command. The database record associated with each command will also contain pointers to the execution of each command. Some non-limiting examples of movement commands may be: “come here,” “follow me,” “forward,” “half speed,” “left,” “reverse,” “right,” and “stop.”

Movement of the robot is controlled by sections of the program associated with each command that calls for movement. Movement is associated or initiated each time by one of: (a) request by parsed verbal command; (b) determination by the program itself as logical consequence of prior or current commands; and (c) locating destination of the movement (e.g., by patient request, by patient signal, such as, waving of the hand etc., by GPS patient micro unit, by command from the control station(s), and by pre-established rules associated with certain emergency conditions). In some embodiments, the grid of GPS locations throughout the patient area may be pre-established beforehand and made a part of the program.

In additional embodiments, the database structure may be dynamic with records created for: (a) creation of entries of events that occur; (b) selection of stored entry as a result of verbal commands by the patient, another user, or control desk; and (c) backup, in the cloud.

As discussed herein, the control program may be written using a decision table structure for the logic, and a decision table translator may generate the actual code for the processor to execute. Accordingly, any command or condition which results in no action is a candidate for learning (i.e., expanding the decision table). Ultimately, all portions of the decision table will be filled out; those that are unknown will be filled from experience. Hence the robotic device is a machine learning device.

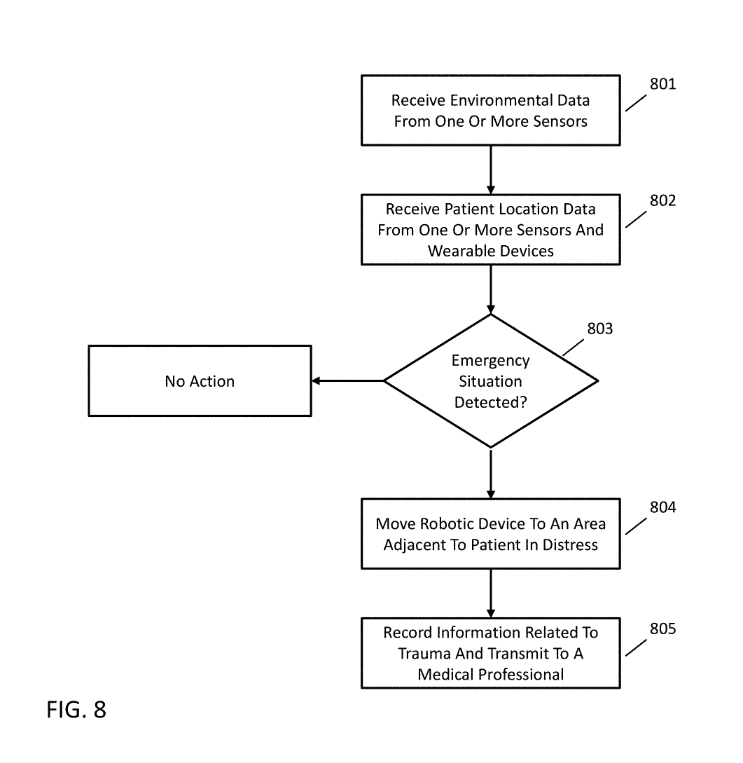

Accordingly, as discussed herein, an embodiment may comprise a robotic companion device with one or more sensors (e.g., as shown in FIG. 1), one or more actuators or actuator devices to facilitate movement, and a network connection device. Referring now to FIG. 8, the robotic device is primarily controlled via a computer-based process which receives various environmental data 801 from the plurality of sensors on board the robotic device. In addition, patient location information is gathered 802 using a combination of integrated sensors and wearable devices. Based on this comprehensive knowledge of both the environment and the patient, the robotic device monitors an area in real time to detect a potential emergency situation 803. As discussed herein, an emergency situation may be detected based on one or more vital signs of the patient exceeding a threshold. This threshold may be predetermined based on information from medical professionals, Internet sources, or client preference. In an additional embodiment, the threshold may be determined by the robotic device based on the historical vital signs previously measured and a variance from a determined statistical mean indicating danger.

In some embodiments, the emergency may be determined based on sensor data (e.g., detecting a “thud” or other stimuli that could indicate the patient has fallen or experienced some form of trauma). In another embodiment, the emergency may be determined based on direct input from the patient (e.g., calling for help, waving their arm, pressing an alarm button, etc.). In yet another embodiment, the emergency may be determined based on similar input from a third party, such as another individual (user) in the area.

Once the emergency situation is recognized, the robotic device may move, using the one or more actuators, to an area adjacent to the patient 804. This movement may be based on known data about the environment as discussed in detail herein. Once the robotic device reaches the patient, it may, as discussed herein, record the vital signs of the patient. In some embodiments, the vital signs may be transmitted to a third party (e.g., medical professional) 805. Additionally, in some embodiments, the robotic device may take images and/or videos of the patient to transmit to a third party 805. Not only can the robotic device send transmission related to the emergency situation, but it may also receive direct instructions from a third party (e.g., medical professional) to relay to the patient via the display device and/or speaker. In an even further embodiment, the robotic device may act as a communication bridge between the patient and third party during the emergency situation.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++ or the like, and conventional procedural programming languages, such as the “C” programming language or similar programming languages. The computer readable program instructions may execute entirely on the robotic device's processor, partly on the robotic device's processor, as a stand-alone software package, partly on the robotic device's processor and partly on a remote processor, or entirely on a remote processor or server. In the latter scenario, the remote computer may be connected to the robotic device's processor through any type of network, including LAN or WAN, or the connection may be made to an external computer (for example, through the Internet using an Internet Service Provider).

Aspects of the present invention are described herein with reference to flowchart illustrations and/or block diagrams of methods, apparatuses (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified herein. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified herein.

The computer readable program instructions may also be loaded onto a computer, other programmable data processing apparatus, or other device to cause a series of operations to be performed on the computer, other programmable apparatus, or other device to produce a computer implemented process, such that the instructions which are executed on the computer, other programmable apparatus, or other device implement the functions/acts specified herein.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical functions. In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

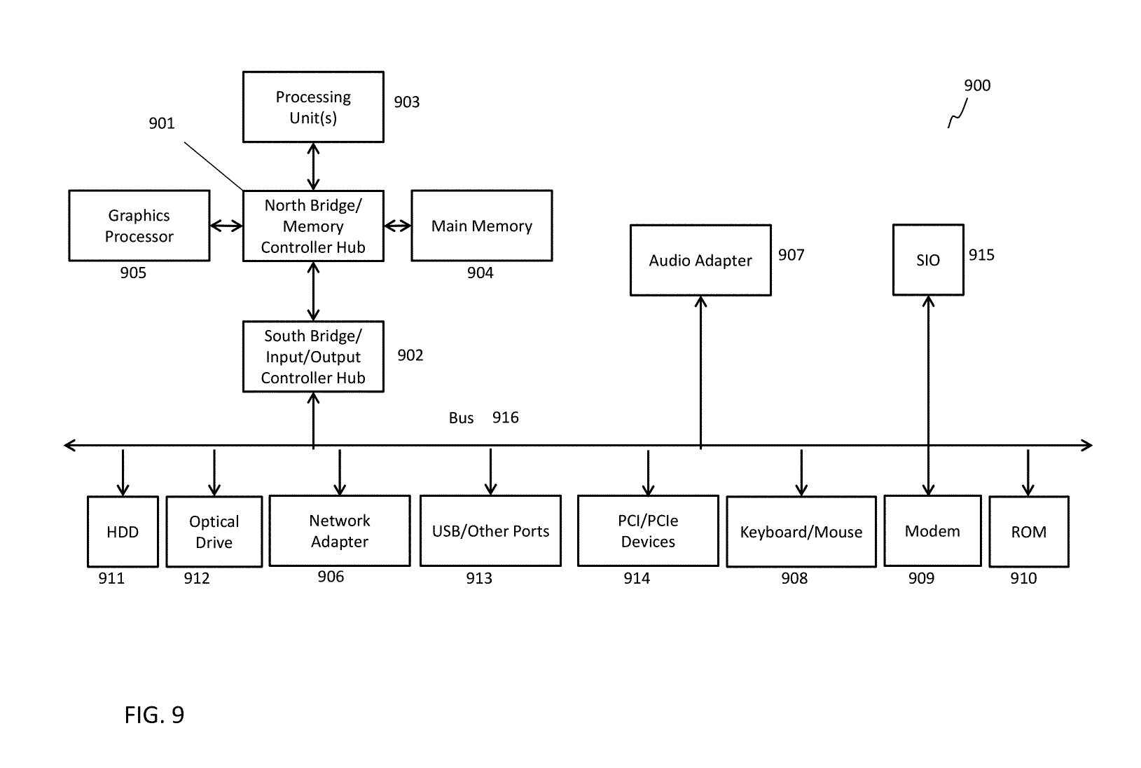

FIG. 9 is a block diagram of an example data processing system 900 in which aspects of the illustrative embodiments are implemented. Data processing system 900 is an example of a computer, such as a server or client, in which computer usable code or instructions implementing the process for illustrative embodiments of the present invention are located. In one embodiment, FIG. 9 may represent a server computing device.

In the depicted example, data processing system 900 can employ a hub architecture including a north bridge and memory controller hub (NB/MCH) 901 and south bridge and input/output (I/O) controller hub (SB/ICH) 902. Processing unit 903, main memory 904, and graphics processor 905 can be connected to the NB/MCH 901. Graphics processor 905 can be connected to the NB/MCH 901 through, for example, an accelerated graphics port (AGP).

In the depicted example, a network adapter 906 connects to the SB/ICH 902. An audio adapter 907, keyboard and mouse adapter 908, modem 909, read only memory (ROM) 910, hard disk drive (HDD) 911, optical drive (e.g., CD or DVD) 912, universal serial bus (USB) ports and other communication ports 913, and PCl/PCIe devices 914 may connect to the SB/ICH 902 through bus system 916. PCl/PCIe devices 914 may include Ethernet adapters, add-in cards, and PC cards for notebook computers. ROM 910 may be, for example, a flash basic input/output system (BIOS). The HDD 911 and optical drive 912 can use an integrated drive electronics (IDE) or serial advanced technology attachment (SATA or eSATA) interface. A super I/O (SIO) device 915 can be connected to the SB/ICH 902.

An operating system can run on processing unit 903. The operating system can coordinate and provide control of various components within the data processing system 900. As a client, the operating system can be a commercially available operating system. An object-oriented programming system, such as the Java programming system, may run in conjunction with the operating system and provide calls to the operating system from the object-oriented programs or applications executing on the data processing system 900. As a server, the data processing system 900 may run the Advanced Interactive Executive operating system or the Linux operating system. The data processing system 900 can be a symmetric multiprocessor (SMP) system that can include a plurality of processors in the processing unit 903. Alternatively, a single processor system may be employed.

Instructions for the operating system, the object-oriented programming system, and applications or programs are located on storage devices, such as the HDD 911, and are loaded into the main memory 904 for execution by the processing unit 903. The processes for embodiments described herein can be performed by the processing unit 903 using computer usable program code, which can be located in a memory such as, for example, main memory 904, ROM 910, or in one or more peripheral devices.

A bus system 916 can be comprised of one or more busses. The bus system 916 can be implemented using any type of communication fabric or architecture that can provide for a transfer of data between different components or devices attached to the fabric or architecture. A communication unit, such as the modem 909 or the network adapter 906 can include one or more devices that can be used to transmit and receive data.

Those of ordinary skill in the art will appreciate that the hardware depicted in FIG. 9 may vary depending on the implementation. Other internal hardware or peripheral devices, such as flash memory, equivalent non-volatile memory, or optical disk drives may be used in addition to or in place of the hardware depicted. Moreover, the data processing system 900 can take the form of any of a number of different data processing systems, including but not limited to, client computing devices, server computing devices, tablet computers, laptop computers, telephone or other communication devices, personal digital assistants, and the like. Essentially, data processing system 900 can be any known or later-developed data processing system without architectural limitation.

The system and processes of the figures are not exclusive. Other systems, processes, and menus may be derived in accordance with the principles of embodiments described herein to accomplish the same objectives. It is to be understood that the embodiments and variations shown and described herein are for illustration purposes only. Modifications to the current design may be implemented by those skilled in the art, without departing from the scope of the embodiments. As described herein, the various systems, subsystems, agents, managers, and processes can be implemented using hardware components, software components, and/or combinations thereof. No claim element herein is to be construed under the provisions of 35 U.S.C. 112(f) unless the element is expressly recited using the phrase “means for.”

Although the invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as they fall within the true spirit and scope of the invention.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, et cetera” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera As a non-limiting example, each range discussed herein can be readily broken down into a lower third, middle third and upper third, et cetera As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Variations of the above-disclosed and other features and functions, or alternatives thereof, may be combined into many other different systems or applications. Variations presently unforeseen or unanticipated alternatives, modifications, variations or improvements therein may be subsequently made by those skilled in the art, each of which is also intended to be encompassed by the disclosed embodiments.

What is claimed is:

1. A robotic companion device comprising:
one or more sensors;
one or more actuators;
a network connection device; and
a non-transitory, processor-readable storage medium that stores instructions executable by a processor to:
receive, from the one or more sensors, sensor data, the sensor data comprising environmental information and patient location information,
determine, based on the sensor data, that an emergency situation has occurred related to one or more patients,
move, using the one or more actuators, the robotic companion device to an area adjacent to the one or more patients, wherein a path to the area is based on the patient location information and the environmental information, and
transmit, using the network connection device, a communication to a third party.

2. The robotic companion device of claim 1, wherein the one or more sensors comprise a sensor selected from the group consisting of proximity sensors, touch sensors, audio sensors, olfactory sensors, light sensors, and visual sensors.

3. The robotic companion device of claim 1, further comprising at least one medical instrument, wherein the instructions are further executable by the processor to:
record, using the at least one medical instrument, vital signs of at least one patient of the one or more patients; and
transmit, using the network connection device, the vital signs to the third party,
wherein the at least one medical instrument comprises a medical instrument selected from the group consisting of an electrocardiogram machine, ophthalmoscope, stethoscope, thermometer, stopwatch, scale, and blood pressure gauge.

4. The robotic companion device of claim 1, wherein the robotic companion device is configured to visually imitate an individual known by the one or more patients, the visual imitation comprising mimicking at least one of gender, voice tone, voice pitch, lip movement, facial appearance, body temperature.

5. The robotic companion device of claim 1, further comprising a mobility base, wherein the mobility base is at least one of: a motorized wheelchair, a telescoping motorized pedestal, and a plurality of motorized limbs.

6. The robotic companion device of claim 1, further comprising at least one image capture device, wherein the instructions are further executable by the processor to:
capture, using the at least one image capture device, at least one image of the one or more patients; and
transmit, using the network connection device, the at least one image to the third party.

7. The robotic companion device of claim 1, wherein the emergency situation is determined based on at least one of:
a vital sign of at least one patient of the one or more patients exceeding a threshold;
the sensor data comprising an indicator that at least one patient of the one or more patients has fallen;
the sensor data comprising an input from at least one patient of the one or more patients indicating an emergency; and
the sensor data comprising an input from one or more users indicating an emergency.

8. The robotic companion device of claim 1, further comprising a display device, wherein the instructions are further executable by the processor to:
display, on the display device, information to the one or more patients.

9. The robotic companion device of claim 1, further comprising at least one medical instrument, wherein the instructions are further executable by the processor to:
store, using the network connection device, medical history records for the one or more patients from one or more medical treatment facilities on the storage medium;
capture, using the at least one medical instruments, vital signs for the one or more patients at regular intervals;
record, on the storage medium, the captured vital signs in the medical history records, and
analyze the medical history records to determine an expected baseline for the vital signs of at least one patient of the one or more patients;
wherein the emergency situation is determined based the one or more patients vital signs exceeding a variation threshold from the expected baseline.

10. The robotic companion device of claim 1, wherein the instructions are further executable by the processor to:
determine, using a decision table, at least one of an autonomous action and a semi-autonomous action, wherein the decision table is generated based on previous interactions with the one or more patients.

11. A robotic companion device comprising:
one or more sensors;
one or more actuators; and
a non-transitory, processor-readable storage medium that stores instructions executable by a processor to:
receive, from the one or more sensors, sensor data, the sensor data comprising environmental information and patient command information, the patient command information comprising a user input, wherein the user input is analyzed to determine that the user is a patient of one or more patients,
determine, based on the patient command information, that an order has been given,
move, using the one or more actuators, the robotic companion device to an area, wherein a path to the area is based on the environmental information, and
perform the order given;
wherein the robotic companion device is configured to visually imitate an individual known by the one or more patients.

12. The robotic companion device of claim 11, wherein the one or more sensors comprise a sensor selected from the group consisting of proximity sensors, touch sensors, audio sensors, olfactory sensors, light sensors, and visual sensors.

13. The robotic companion device of claim 11, further comprising a network connection device, wherein the instructions are further executable by the processor to:
establish, using the network connection device, a line of communication with at least one of a third party or third-party device in order to perform the given order.

14. The robotic companion device of claim 11, further comprising at least one image capture device, wherein the instructions are further executable by the processor to:
capture, using the at least one image capture device, at least of an image and video in order to perform the given order.

15. The robotic companion device of claim 14, further comprising at least one speaker, wherein the instructions are further executable by the processor to:
capture, using the at least one image capture device, an image containing text;
analyze the image using optical character recognition to identify the text;
emit, using the at least one speaker, the identified text in order to perform the given order.

16. The robotic companion device of claim 11, further comprising at least one display device, and a network connection device, wherein the instructions are further executable by the processor to:
receive, using the network connection device, data from a third party;
display, on the display device, the data from a third party in order to perform the given order;
wherein the data from a third-party comprises at least one of a digital image, a digital video, and text.

17. The robotic companion device of claim 11, further comprising at least one touch display device, and a network connection device, wherein the instructions are further executable by the processor to:
receive, using the at least one touch display, user input; and
display, on the display device, a website based on the user input in order to perform the given order.

18. The robotic companion device of claim 11, further comprising a mobility base, wherein the mobility base is at least one of: a motorized wheelchair, a telescoping motorized pedestal, and a plurality of motorized limbs.

19. A system comprising:
a robotic companion device comprising:
one or more sensors,
one or more actuators,
a network connection device,
a first power connector, and
a non-transitory, processor-readable storage medium that stores instructions executable by a processor to:
receive, from the one or more sensors, sensor data, the sensor data comprising environmental information and patient location information,
determine, based on the sensor data, that an emergency situation has occurred related to one or more patients,
move, using the one or more actuators, the robotic companion device to an area adjacent to the one or more patients, wherein a path to the area is based on the patient location information and the environmental information, and
transmit, using the network connection device, a communication to a third party; and
a base station comprising:
a second power connector, and
a power source,
wherein the instructions are further executable by the processor to establish a connection between the first power connector and the second power connector, and wherein power is transferred from the power source to the robotic companion.

20. The robotic companion device of claim 19, wherein the connection between the first power connector and the second power connector is at least one of a wired connection and a wireless connection.
